(19) United States
(12) Patent Application Publication
(10) Pub. No.: US 2025/0120668 A1
(43) Pub. Date: Apr. 17, 2025

(54) Test body for checking image quality during an X-ray inspection of a test object, use of such a test body, and method for determining the defect detection rate and/or for determining the depth of field of an X-ray system

(71) Applicant: Comet Yxlon GmbH, Hamburg (DE)

(72) Inventor: André Schu, Hamburg (DE)

(21) Appl. No.: 18/915,769

(22) Filed: Oct. 15, 2024

(30) Foreign Application Priority Data
Oct. 16, 2023 (DE) 10 2023 128 240.9

(51) Int. Cl. A61B 6/58; G06T 7/00

(52) U.S. Cl. CPC A61B 6/583; G06T 7/0012

(57) ABSTRACT

A test body for checking image quality during an X-ray examination of a test object that has a first layer of test subjects to be checked and has an intermediate layer on which the test subjects are arranged is disclosed. The test body has a first test layer and a solid spacer plate arranged thereon. The thickness of the first test layer corresponds to the thickness of the test subjects in the test object, and the thickness of the spacer plate corresponds to the thickness of the intermediate layer. A plurality of first holes are formed in the first test layer. The material of the first test layer has an absorption factor that corresponds to the absorption factor of the associated test subjects. The material the spacer plate has an absorption factor that corresponds to the absorption factor of the intermediate layer.

### PRIORITY CLAIM

The present application claims priority under 35 U.S.C. § 119 to German Patent Application No. DE 10 2023 128 240.9, filed Oct. 16, 2023, the disclosure of which is incorporated by reference herein in its entirety.

### TECHNICAL FIELD

The present disclosure relates to a test body for checking the image quality during an X-ray inspection of a test object that has multiple layers of test subjects to be checked that are the same size in each individual layer and has an intermediate layer between these respective layers, and to the use of such a test body for determining the defect detection rate and/or for determining the depth of field of an X-ray system. It also relates to a method for determining the defect detection rate and/or for determining the depth of focus of an X-ray system.

### DESCRIPTION OF STATE OF THE ART

The area of application for the present disclosure is X-ray-based materials testing. The use of X-rays for imaging offers the possibility of examining hidden structures without destroying the test object. The inspection is carried out in an X-ray system that has an X-ray tube (hereinafter referred to as “tube”) and an X-ray detector (hereinafter referred to as “detector”) as the imaging system. The test object to be inspected is arranged therebetween on a manipulator. Some or all of the three aforementioned components can be moved translationally and/or rotationally, depending on the X-ray system. The entire device is located in a radiation protection cabin. In many systems, the inspection of test objects is carried out manually. The operator must independently check the image quality at regular intervals and ensure that the image chain (X-ray tube, manipulator, X-ray detector) is able to image the defect sizes being sought. For better reproducibility, and to increase the level of automation during inspection, an automatic check of the image chain by the system is necessary. A test object is required that can be regularly moved into the image and on which the system can evaluate whether the defect sizes sought can still be reproduced in the test object or whether an adjustment of the system parameters is necessary in order to restore the desired state. Up to now, such a check has been performed, for example, by inserting resolution samples and adjusting the focus of the X-ray tube based on the image.

The focus size is a parameter that affects the quality of laminography scans. However, there are also other parameters, such as precision of manipulation, object movement, or adjustment.

U.S. Pat. No. 6,694,047 B1 and EP 0 874 536 A1 describe test bodies that are intended to cover many different applications in a single test body. Hole-type penetrameters with different filter thicknesses, line pairs, and step wedges are accommodated on a test body. The regions on the test body that are to be used must then be selected depending on the application, and the quality of the image—for example the signal-to-noise ratio (SNR) or the contrast-to-noise ratio (CNR)—can then be evaluated for a position in the test volume. If this is to be done for multiple regions of the test volume, then the measurements have to be performed one after the other and the test body positioned differently.

The ASTM E 2737 standard also describes the image quality at a few points in the test space. Here, too, hole-type penetrameters and step wedges are used to simulate the actual application and, for example, to evaluate the SNR and CNR. For the sake of example, the procedure would be as follows: If a test object is made of aluminum and has a maximum penetration length of 100 mm, 100 mm of aluminum would be used as the filter. If a porosity of 1 mm is to be made visible in this aluminum, a hole-type penetrameter with the corresponding hole size would be mounted on the filter. If the hole can be detected in the X-ray image, it can be assumed that a porosity of this magnitude can also be detected.

One major drawback of previous solutions is that the image quality is only checked at one point in the image. Although it is possible to move the test bodies to different points and check the quality at multiple points one after the other, this is very time-consuming. The longer such an image quality check lasts, the less meaningful such a check becomes, since the image quality can change due to thermal drifts, for example. Suppose three points are to be checked in a test volume, and the first two points are OK and meet the specification, but the third point does not meet the specifications. The question now is whether the first two points are still within the specifications or whether the system has changed and the first two points that were checked are now worse. Some rechecking would be required in order to be able to ascertain reliable information about the points. The advantage of checking the three points simultaneously is that different system states can be ruled out as a defect factor.

### SUMMARY OF DISCLOSED EMBODIMENTS

It is the object of the present disclosure to provide a test body with which information can be ascertained about the image quality of the X-ray procedure in all image regions of one plane or of multiple planes.

The object is achieved according to the present disclosure by a test body with the features described herein, a use of the test body with the features described herein, and a method with the features described herein. Advantageous embodiments are specified in the present disclosure.

Accordingly, the object is achieved by a test body whose structure is similar to the test object that is to be inspected by the X-ray system. The test body according to the present disclosure is used for test objects to be inspected having a layer of test subjects to be checked that are of the same size and that are arranged on a solid spacer plate. The test body has a test layer. The thickness of the test layer corresponds to the thickness of the test subjects in the test object. In order to achieve the best possible imitation of the test object, the test body also has a solid spacer plate—i.e., one made entirely of material without holes or the like-which is arranged on the first test layer, has the same thickness as the intermediate layer, and is made of a material that has an absorption factor corresponding to that of the material of the intermediate layer. In order to assess whether the desired image quality is ensured over the entire surface of the test object, a plurality of first holes are formed in the first test layer whose depth and diameter are the same and whose size corresponds to a fraction of the size of the associated test subjects that are to be detected as defects. The material from which the first test layer is made has an absorption factor that corresponds to the absorption factor of the test subjects, which ensures that the absorption of X-ray radiation approximates that in the test object as closely as possible. One advantage of the simultaneous inspection of different points/regions in the entire X-ray beam, made possible by the plurality of first holes, is that different system states can be ruled out as a defect factor.

One advantageous refinement of the present disclosure makes a provision that the following additional features are present if the test object has a second layer of test subjects to be checked:

The test body has a second test layer that is arranged on the side of the spacer plate facing away from the first test layer, wherein the thickness of the second test layer corresponds to the thickness of the test subjects in the second layer and the second test layer is made of a material that has an absorption factor that corresponds to that of the material of the test subjects of the second layer, wherein a plurality of first holes are formed in the second test layer whose depth and diameter are equal and whose size corresponds to the fraction of the size of the associated test subjects that is to be detected as a defect. The quality of the depth of field—i.e., whether at different depths that correspond to the levels of the two test layers—can be easily assessed, since the combination of the first test layer, spacer plate, and second test layer of the test body correspond in their respective thicknesses to the first layer, intermediate layer, and second layer of the test object and are each made of a material with a corresponding absorption coefficient, meaning that the same absorption of X-rays occurs as in the test object, and the second test layer of the test body is arranged at the same location in the beam path as the layer with the test subjects to be inspected in the test object.

Another advantageous refinement of the present disclosure makes a provision that the test body has at least one additional combination of an additional spacer plate and an additional test layer, the additional spacer plate and the additional test layers each corresponding to the spacer plate and second test layer according to the preceding paragraph. This also makes it possible to simulate test objects for testing using a test body that have a large number of test layers-possibly even with test subjects that are the same size within a layer but may also be of different sizes in different layers.

Another advantageous refinement of the present disclosure makes a provision that second holes are formed in each test layer that have at least twice the diameter of the first holes and the same depth as the first holes. This makes it easy to determine the CNR.

Another advantageous refinement of the present disclosure makes a provision that third holes are formed in each test layer that have at least quadruple the diameter of the first holes, the third holes having the same depth as the first holes. This also makes it easy to determine the CNR.

Another advantageous refinement of the present disclosure makes a provision that the first holes and/or the second holes and/or the third holes are each arranged in at least one of the test layers in a matrix that extends over a large portion of the surface of the test layer, all holes being particularly arranged in a common matrix. Due to the large-area distribution of holes, the test body does not have to be positioned at a large number of locations in order to determine the image quality over the surface of the test object, but rather this can be achieved by a single position that corresponds to that of the test object during the inspection. This saves time and prevents possible inaccuracies when positioning the test body multiple times at different points in the beam path.

Another advantageous refinement of the present disclosure makes a provision that the test object is a circuit board and the test subjects are solder balls. These are the most common applications within the scope of the present disclosure.

In such a case, it is preferred that the test layers be made of a material with a higher atomic number (in the context of this application, this is understood to mean an atomic number greater than 22) such as nickel, tin, or copper, or an alloy containing at least one of these substances, and/or that the spacer plates be made of a material with a lower atomic number (in the context of this application, this is understood to mean an atomic number less than 14), preferably silicon. These are the materials of the test subjects to be inspected that are used in the most common applications within the scope of the present disclosure.

The object is also achieved by the use of a test body according to the present disclosure for determining the defect detection rate and/or for determining the depth of field of an X-ray system with a tube, a detector, and a holder arranged therebetween for receiving the test object.

Finally, the object is also achieved by a method for determining the defect detection rate and/or for determining the depth of field of an X-ray system with a tube, a detector, and a holder arranged therebetween for receiving the test object, in that a single X-ray image is taken of a test body according to the present disclosure that is located in the beam path instead of the test object at the location where the test object is located during the inspection, and this X-ray image is then evaluated.

### BRIEF DESCRIPTION OF THE FIGURES

Further details and advantages of the present disclosure will now be explained in greater detail with reference to exemplary embodiments illustrated in the drawings, in which:

FIG. 1 shows a schematic representation of a section of a test layer with holes in plan view.

FIG. 2 shows a part of a schematic longitudinal section through a test layer with holes.

FIG. 3 shows an isometric view of three test layers arranged one above the other according to FIG. 1.

FIG. 4 shows a part of a schematic longitudinal section through a test body with two test layers with interposed spacer plate.

FIG. 5 shows a comparison of a schematically illustrated test object in longitudinal section with an associated test body in a schematic representation in longitudinal section.

FIG. 5A shows an enlarged representation of the test test body according to FIG. 5.

### DETAILED DESCRIPTION OF EXEMPLARY EMBODIMENTS

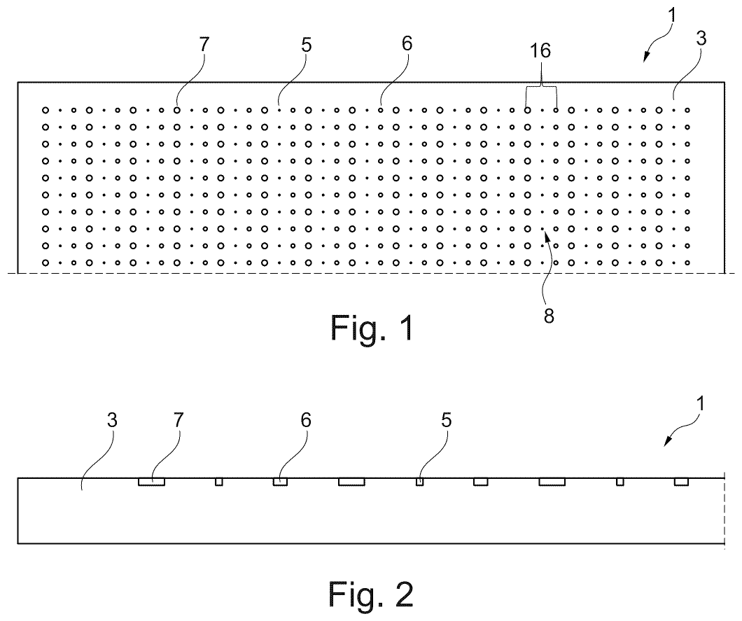

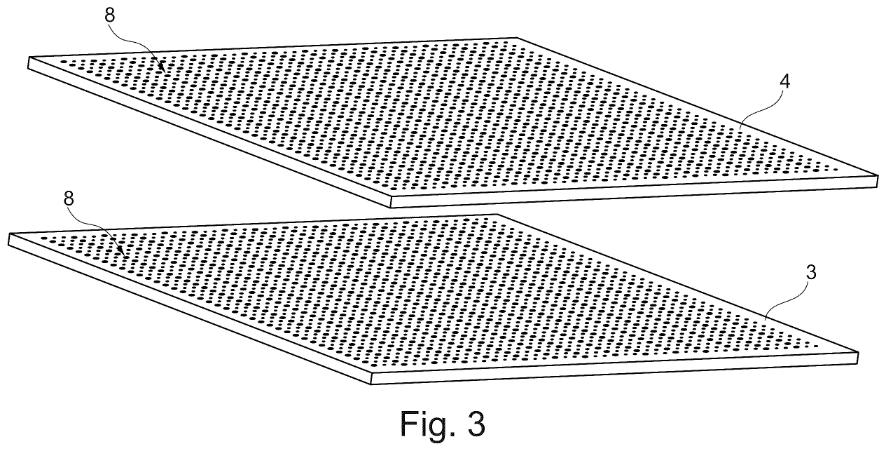

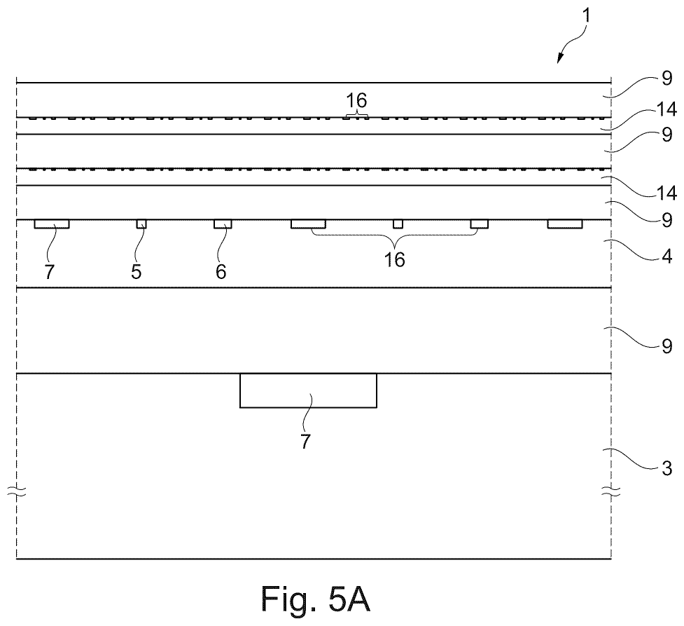

FIGS. 1 to 4 show parts of a test body 1 and the entire test body 1 for an application that is associated with a test object 2, the structure of which is as follows: It is a circuit board with two levels in which test subjects 10 to be inspected are present. In the first level, there is a ball grid array (BGA) with balls (solder balls) of size 200 μm (diameter). The second level is arranged at a distance of 300 μm and includes a BGA with 20 μm solder balls. Between the two levels, there is a substrate made of silicon (corresponding to the intermediate layer 15 shown in FIG. 5). It should be possible to detect voids (air inclusions in the balls) of 10% of the ball size in each plane.

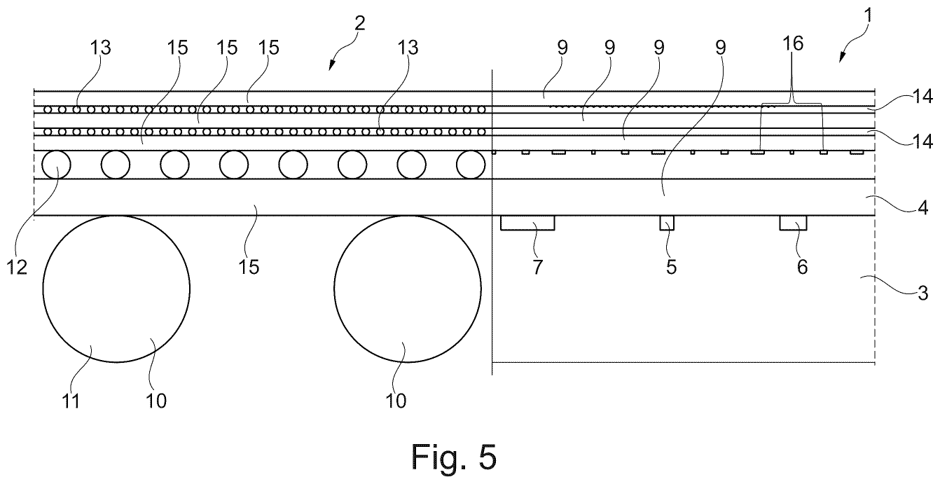

FIG. 1 shows a schematic representation of a section of a first test layer 3 according to some embodiments of a test body 1 according to some embodiments (see FIGS. 4 and FIGS. 5 and 5A for another embodiment) in plan view. The first test layer 3 is made of the same material as the test subjects 10 (e.g., solder balls) of the first level in the test object 2 (shown in FIG. 5 for the other exemplary embodiment) with which the test body 1 is associated. The association means that the test object 1 is used to check the image quality during series inspection of identical test objects 2 and is structurally replicated. In the present case, the first test layer 3 is made of tin, since the test objects 10 in the lower level of the test object 2 are solder balls 10 made of tin.

Holes 5, 6, 7 are formed in the surface of the first test layer 3 and are arranged in a square matrix 8 in the exemplary embodiment shown. There are three different types of hole: first holes 5 (also called T1 holes), second holes 6 (also called T2 holes), and third holes 7 (also called T4 holes). Their respective diameters and depths depend on the size of the test subjects 10 corresponding to them and the size of any defects in them that are to be detected. The diameter of the first holes 5 is as large as the defect that is still to be detected. In the present case, as stated above, this is 10% of 200 μm, or 20 μm. The depth of the first holes 5 is equal to their diameter, i.e., also 20 μm. The assumption here is that if the T1 hole (first hole 5) is detectable in the image, a 10% void in a solder ball 10—the test subject 10—would also be detectable.

The second holes 6 (T2 hole) have twice the diameter of the first holes 5, i.e., 40 μm, and the third holes 7 have quadruple the diameter of the first holes 5, i.e., 80 μm. The depth of these holes 6, 7 is the same as the depth of the first holes 5, i.e., 20 μm. These holes 6, 7 are used to determine a CNR.

An ensemble of three adjacent holes 5, 6, 7 in a row (this is shown as an example in FIG. 1 for the first row) represents a hole pentrameter 16 that is known from the prior art. In the prior art (see also the abovementioned U.S. Pat. No. 6,694,047 B1 and EP 0 874 536 A1), a test body 1 has only one hole-type penetrameter 16 or a small number of hole-type penetrameters 16—for example three. By virtue of the large number of hole-type penetrameters 16 in the test body 1 according to some embodiments in a large number of rows of the matrix 8—distributed over the entire surface of the first test layer 3—it is sufficient to take a single X-ray image of the test body 1 only at one point (corresponding to that of the test object 2 during the inspection) in order to be able to determine the image quality, SNR, and CNR. This greatly reduces the time required to qualify the test space. In anticipation of the explanations which follow regarding the second test layer 4 and the spacer plate 9 located between the two test layers 3, 4, this is also true with regard to the position in the beam path along the X-ray beam.

Since the second level of the test object 2 has solder balls to be inspected with a size (diameter) of 40 μm, the second test layer 4 also has a material thickness of 40 μm. Since a defect of 10% is to be detected, the first holes 5 have a diameter of 4 μm; accordingly, the second holes 6 have a diameter of 8 μm, and the third holes 7 have a diameter of 16 μm. The hole depth of all of the holes 5, 6, 7 is 4 μm. This second test layer 4 is spaced apart from the first test layer 3 by a spacer plate 9 with a thickness of 50 μm. In order to approximate the actual application as closely as possible, the spacer plate 9 is made of a 50 μm-thick silicon disc. In principle, any material that approximates that of the application can be used for the spacer plate 9; if possible, it should have the same absorption coefficient as the corresponding material of the test object 2 or be so thick that its absorption on the beam path through it approximately corresponds to the absorption in the beam path between the two planes of the test object 2. The entire structure can be seen in FIG. 4 in a schematic, partial longitudinal section.

By stacking and adjusting the actual distances of the layers and components of test object 2—here a BGA—to the focus of the X-ray tube, it is possible to detect the depth of field of the system. This is not constant for the test space. It is therefore important to know the structure of the circuit board in order to be able to check whether defect sizes can still be detected at the actual distances.

Since both the size of the holes 5, 6, 7 and the arrangement of the test layers 3, 4, 14 must be flexibly adapted to the application—i.e., the test object 2 to be inspected—a general description of the test body 1 is difficult. Reference is therefore made to the explanation above in relation to an example application. In the following, the structure of a test body 1 according to some embodiments will be explained on the basis of another exemplary embodiment according to FIGS. 5, 5A. In FIG. 5, the test object 2 to be inspected is shown on the left side, and the test body 1, which is a replica thereof, is shown on the right side. In FIG. 5A, the test body 1 according to FIG. 5 is shown under enlargement in order to facilitate recognition of the holes 5, 6, 7 in particular.

The example test object 2 is a circuit board with different levels in which different components are present. In the following, the structure thereof is discussed from bottom to top, with only the components that are essential to the embodiments being discussed.

In the lowest level, the test subjects 10 are a large number of solder balls 10 in the form of package balls 11 with a diameter of 200 μm, which are usually made of tin or an alloy with a high tin content. An intermediate layer 15 is arranged above this. This is followed by a further level with test subjects 10 in the form of a large number of solder balls 10 (here: C4 bumps 12, which are usually made of tin or an alloy with a high tin content, with a diameter of 40 μm). Another intermediate layer 15 is applied on top of this. On this intermediate layer 15, there is another level of test subjects 10 in the form of a large number of solder balls 10 (here: microbumps 13, which are usually also made of tin or an alloy with a high tin content, with a diameter of 10 μm). The next layer is again an intermediate layer 15. Above this there is another structure that includes the two levels described above: microbumps 13 with intermediate layer 15 on top.

As part of the non-destructive X-ray analysis of such circuit boards, the quality of the solder joints—i.e., of the test subjects 10—between the functional and carrier components of the circuit board is checked (among other things). This is usually done using a laminography process that is well known in the art. It is necessary to regularly check whether the image chain can achieve a sufficiently high image quality of the X-ray images taken using the laminography method. This is also crucial inter alia with regard to whether a defect can be detected in the test subjects 10 whose size exceeds a predeterminable level-which would result in this circuit board having to be removed from the production process. In order to be able to provide such an assessment of the image quality within the scope of the series inspection of a circuit board designed in this way, a test body 1 according to some embodiments is used that reproduces the test object 2 as accurately as possible with regard to its X-ray properties—as was already described above for another embodiment shown in FIGS. 1 to 4. A test body 1 according to some embodiments shown on the right-hand side of FIG. 5 and FIG. 5A, which corresponds to the test object 2 shown on the left-hand side of FIG. 5 and was just described in more detail, is held in the beam path of the X-ray system at the point where the test object 2 is located during the inspection, and a single image is taken of the test body 1. Using this, the information already mentioned above for the other exemplary embodiment regarding the image quality can be ascertained over the entire test region-both in the plane perpendicular to the X-ray beam and in the direction of the X-ray beam (i.e., the depth).

In the following, the structure of the test body 1 associated with the test object 2 in FIG. 5 will be described in order to be enable the aforementioned check of the image quality to be carried out as well as possible with the least possible effort.

The bottom layer, the first test layer 3—which corresponds to the level of the package balls 11 of the test object 2—includes copper with a thickness of 200 μm. There are three types of hole 5, 6, 7 in its surface (as already described above for the other exemplary embodiment of FIGS. 1 to 4): a first hole 5 (T1 hole) with a diameter of 20 μm, a second hole 6 (T2 hole) with double the diameter, and two third holes 7 (T4 hole) with quadruple the diameter; all of the holes 5, 6, 7 have the same depth-corresponding to the diameter of the first hole 5—of 20 μm.

A spacer plate 9 made of silicon with a thickness of 50 μm is arranged on the first test layer 3. It equates to an intermediate layer 15 of the test object 2.

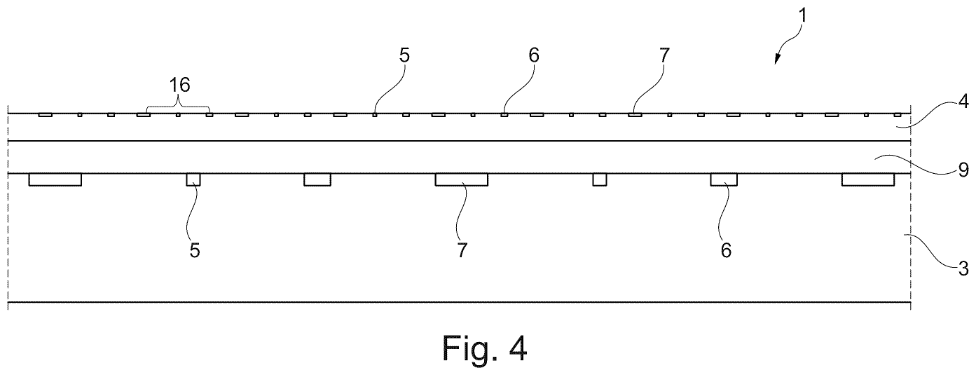

On top of this is a second test layer 4 made of copper with a thickness of 40 μm, which corresponds to the plane of the C4 bumps 12 of the test object 2. There are also three types of hole 5, 6, 7 in its surface (as already described above for the other exemplary embodiment of FIGS. 1 to 4 and the first test layer 3 of the present exemplary embodiment): first holes 5 with a diameter of 4 μm, second holes 6 with double the diameter, and third holes 7 with quadruple the diameter; all of the holes 5, 6, 7 have the same depth of 4 μm, corresponding to the diameter of the first holes 5. The holes 5, 6, 7 arranged in a matrix 8 (only in plan view, as visible in FIGS. 1 and 3) form a plurality of hole-type penetrameters 16, each of which is formed from three different holes 5, 6, 7 lying next to one another (see also FIGS. 1 and 4).

Another spacer plate 9 made of silicon with a thickness of 20 μm is arranged on the second test layer 4. It equates to an intermediate layer 15 of the test object 2.

On top of this is a third test layer 14 made of copper with a thickness of 10 μm, which corresponds to the level of the microbumps 13 of the test object 2. There are also three types of hole 5, 6, 7 in its surface (as already described above for the first test layer 3 and the second test layer 4 of the present embodiment): first holes 5 with a diameter of 1 μm, second holes 6 with double the diameter, and third holes 7 with quadruple the diameter; all of the holes 5, 6, 7 have the same depth of 1 μm, corresponding to the diameter of the first holes 5. These holes 5, 6, 7 are also arranged in a matrix 8, whereby a plurality of hole-type penetrameters 16 are formed.

Another spacer plate 9 made of silicon with a thickness of 20 μm is arranged on the third test layer 14. It equates to an intermediate layer 15 of the test object 2.

On top of this is a fourth test layer 14 made of copper with a thickness of 10 μm, which corresponds to the level of the microbumps 13 of the test object 2. There are also three types of hole 5, 6, 7 in its surface (as already described above for the first test layer 3 and the second test layer 4 of the present embodiment): first holes 5 with a diameter of 1 v, second holes 6 with double the diameter, and third holes 7 with quadruple the diameter; all of the holes 5, 6, 7 have the same depth of 1 μm, corresponding to the diameter of the first holes 5. These holes 5, 6, 7 are also arranged in a matrix 8, whereby a plurality of hole-type penetrameters 16 are formed.

Another spacer plate 9 made of silicon with a thickness of 20 μm is arranged on the fourth test layer 14. It equates to an intermediate layer 15 of the test object 2.

Thus, the present test body 1 provides a device that corresponds exactly to the test object 2 and with which the examination of the image quality during the series inspection of such test objects 2 can be carried out simply and quickly.

### LIST OF REFERENCE SYMBOLS

1 test body
2 test object
3 first test layer
4 second test layer
5 first hole
6 second hole
7 third hole
8 matrix
9 spacer plate
10 test subject, in particular solder ball
11 package ball
12 C4 bump
13 microbump
14 additional test layer
15 intermediate layer
16 hole-type penetrameter

1. A test body for checking an image quality during an X-ray examination of a test object that has a first layer of test subjects to be checked and that has an intermediate layer on which the test subjects are arranged,
wherein the test body has a first test layer and a solid spacer plate arranged thereon,
wherein a thickness of the first test layer corresponds to a thickness of the test subjects in the test object, and a thickness of the spacer plate corresponds to a thickness of the intermediate layer,
wherein a plurality of first holes are formed in the first test layer, whose depth and diameter are equal and whose size corresponds to a fraction of the size of the associated test subjects that are to be detected as defects,
wherein the material of the first test layer has an absorption factor that corresponds to an absorption factor of the associated test subjects, and
wherein the material the spacer plate has an absorption factor that corresponds to an absorption factor of the intermediate layer.

2. The test body according to claim 1, which is associated with a test object having a second layer of test subjects to be inspected that are arranged on a side of the intermediate layer (15) facing away from the first layer,
wherein the test body has a second test layer that is arranged on a side of the spacer plate facing away from the first test layer,
wherein a thickness of the second test layer corresponds to a thickness of the test subjects in the second layer, and the second test layer is made of a material having an absorption factor corresponding to that of a material of the test subjects of the second layer, and
wherein a plurality of first holes are formed in the second test layer whose depth and diameter are equal and whose size corresponds to a fraction of the size of the associated test subjects that are to be detected as defects.

3. The test body according to claim 2, wherein the test body has at least one additional combination of an additional spacer plate and an additional test layer, the additional spacer plates and the additional test layers corresponding respectively to the spacer plate and the second test layer.

4. The test body according to claim 1, wherein second holes are formed in each test layer that have at least twice a diameter of the first holes, and wherein the second holes have a same depth as the first holes.

5. The test body according to claim 4, wherein third holes are formed in each test layer that have at least quadruple a diameter of the first holes, and wherein the third holes have a same depth as the first holes.

6. The test body according to claim 5, wherein the first holes and/or the second holes and/or the third holes are each arranged in at least one of the test layers in a matrix that extends over a large portion of a surface of the test layer, wherein all holes are arranged in a common matrix.

7. The test body according to claim 1, wherein the test object is a circuit board and the test subjects are solder balls.

8. The test body according to claim 1, wherein the test layers are made of a material with an atomic number greater than 22 and/or the spacer plates are made of a material with an atomic number less than 14.

9. A use of a test body according to claim 1 for determining a defect detection rate and/or for determining a depth of field of an X-ray system with a tube, a detector, and a holder arranged therebetween for receiving the test object.

10. A method for determining a defect detection rate and/or for determining a depth of field of an X-ray system with a tube, a detector, and a holder arranged therebetween for receiving the test object according to claim 1,
wherein a single X-ray image is taken of the test body that is located in a beam path instead of the test object at a location where the test object is located during an inspection, and this X-ray image is then evaluated.
